(19) United States
(12) Patent Application Publication
(10) Pub. No.: US 2016/0060190 A1
(43) Pub. Date: Mar. 3, 2016

(54) PROCESS FOR PRODUCING A SWEETENED HYDROCARBON STREAM

(71) Applicant: UOP LLC, Des Plaines, IL (US)

(72) Inventors: Jessy E. Trucko, Lake Forest, IL (US); Steven A. Bradley, Arlington Heights, IL (US); Haiyan Wang, Hoffman Estates, IL (US); Mark W. Mucek, Spring Grove, IL (US); Benjamin Lee Tiemens, Chicago, IL (US); Derek Froehle, Wheeling, IL (US)

(21) Appl. No.: 14/473,162

(22) Filed: Aug. 29, 2014

(51) Int. Cl. C07C 7/00; C07C 7/148; C07C 7/12; C10L 3/10

(52) U.S. Cl. CPC C07C 7/005; C10L 3/102; C07C 7/14841; C07C 7/12; C10L 2290/08

(57) ABSTRACT

A process of producing a sweetened liquid hydrocarbon stream. In order to prevent the forming of acid species in a sweetening zone, a oxygenate removal zone is disposed upstream of the sweetening zone. The oxygenate removal zone may comprise a water wash or an adsorbent zone, including a regenerable adsorbent. The sweetened stream is formed from a least a portion of liquid natural gas stream which may be scrubbed free of hydrogen sulfide and dehydrated before passing to the oxygenate removal zone.

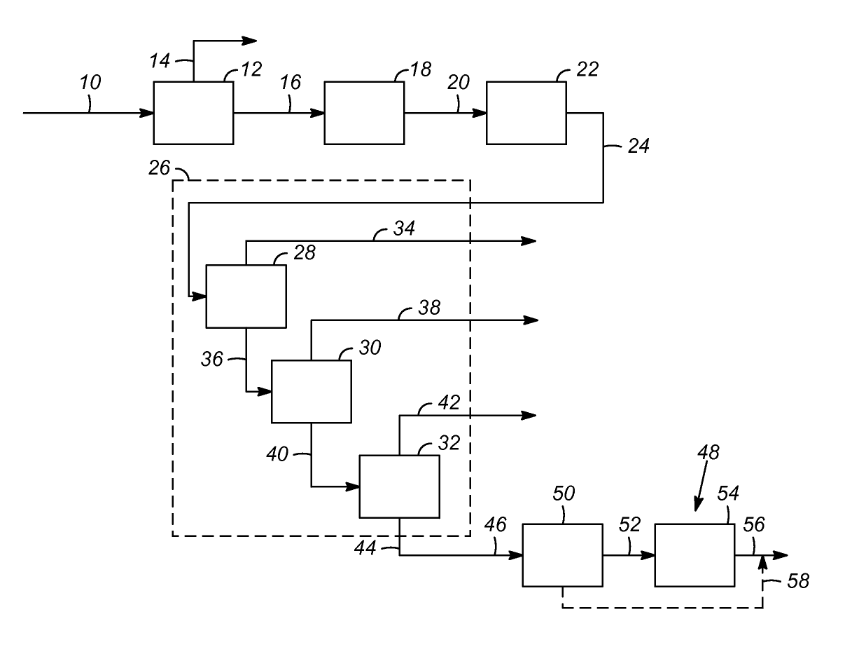

### FIELD OF THE INVENTION

This invention relates generally to process for producing a sweetened hydrocarbon stream and more particular to a process for minimizing corrosion in a reactor that converts the mercaptans in a hydrocarbon stream into disulfides.

### BACKGROUND OF THE INVENTION

Natural gas processing processes frequently involve removal of organic sulfur compounds. The removal of the organic sulfur compounds, as well as other contaminants, is required to meet end-product specifications and to avoid product blockages in downstream process equipment. The level of treatment that is required varies according to the end-product specifications, as well as local environmental regulations.

For example, a mercaptan conversion may be performed in one or more vessels that contain a catalyst. One such process is described in U.S. Pat. No. 4,124,493, the entirety of which is incorporated herein by reference. Many times these vessels can be comprised of a carbon steel material. Often, the catalyst in these vessels comprises cobalt on a support, such as activated carbon.

Recently, accelerated corrosion in multiple locations within various mercaptan conversion vessels has been observed. Additionally, large portions of catalyst materials in the mercaptan conversion vessels have been observed as having agglomerated. Upon investigation, it was discovered that an increased amount of acidic species was present in the mercaptan conversion vessels.

It is believed that the acid species in the mercaptan conversion vessels are the result of various acid precursors, such as one or more oxygenates, reacting with the cobalt in the catalyst. The oxygenates may include one or more of glycols, poly glycols, organic acids, aldehydes, ketones, ethers, esters and alcohols. For example one such class of oxygenates that may be reacting to form acids is glycols.

Glycol is typically used in such processes upstream of the mercaptan conversion vessel, typically, in a dehydration unit (or glycol dryer). More specifically, a glycol dryer is typical disposed after a water wash of a stream from an amine absorber. Thus, glycol may be present in the feed stream as a result of the upstream dehydration unit.

Other acid precursors may be present in the feed stream as a result of one or more processes used to enhance the recovery of natural gas from a gas well.

Therefore, it would be desirable to have one or more processes which minimize corrosion associated with forming acidic species in the mercaptan conversion vessels.

### SUMMARY OF THE INVENTION

One or more processes have been invented in which oxygenates, such as glycol, are removed from a portion of a liquid natural gas stream upstream of a sweetening zone in order to minimize corrosion associated with acidic species in a reactor of the sweetening zone.

A first aspect of the invention may be characterized as a process for producing a sweetened hydrocarbon stream in which the process comprises: passing a liquid hydrocarbon stream to an oxygenate removal zone; removing oxygenates from the liquid hydrocarbon stream in the oxygenate removal zone to form an oxygenate lean stream; and, passing the oxygenate lean stream to a sweetening zone to convert organic mercaptan in the oxygenate lean stream to disulfide and produce a sweetened hydrocarbon steam.

In some embodiments of the present invention, the oxygenates comprise at least one of glycols, poly glycols, organic acids, aldehydes, ketones, ethers, esters and alcohols, and the oxygenate removal zone comprises a water wash.

In some embodiments of the present invention, the oxygenate removal zone comprises an adsorbent zone. It is contemplated that the adsorbent zone comprises a regenerable adsorbent. It is contemplated that the process also includes the steps of desorbing oxygenates from the regenerable adsorbent to provide a desorbent stream rich in oxygenates and combining the desorbent stream rich in oxygenates with the sweetened hydrocarbon stream.

In at least one embodiment, the sweetening zone includes a catalyst. It is contemplated that the catalyst includes cobalt on a support.

In some embodiments, the liquid hydrocarbon stream comprises a bottoms stream from a separation zone.

In at least one embodiments, the liquid hydrocarbon stream comprises a C₅+ hydrocarbon stream.

In some of the embodiments of the present invention, the stream passed into the mercaptan conversion zone is a liquid hydrocarbon stream that is lean in disulfides.

A second aspect of the invention may be characterized as a process for producing a sweetened hydrocarbon stream in which the process comprises: removing water from a liquid hydrocarbon stream in a glycol dehydration zone; passing at least a portion of the liquid hydrocarbon stream from the dehydration zone to an oxygenate removal zone; removing oxygenates from the liquid hydrocarbon stream in the oxygenate removal zone to form an oxygenate lean stream; and, passing the oxygenate lean stream to a sweetening zone to reduce an amount of mercaptans in the oxygenate lean stream and produce a sweetened hydrocarbon stream. The sweetening zone comprises at least one vessel, each vessel containing a catalyst.

In at least one embodiment, each vessel of the sweetening zone comprises at least portion of a carbon steel material. It is contemplated that the catalyst in each vessel of the sweetening zone comprises cobalt.

In some embodiments of the present invention, the process includes removing acid gas from the liquid hydrocarbon stream upstream of the sweetening zone. It is contemplated that the oxygenate removal zone comprises a water wash. It is also contemplated that the oxygenate removal zone comprises an adsorbent zone. It is further contemplated that the adsorbent zone comprises a regenerable adsorbent and the process may further comprise regenerating the regenerable adsorbent. It is still further contemplated that the process includes separating the hydrocarbon stream after the step of removing acid gas in a separation zone into at least a C₅+ hydrocarbon stream. The C₅+ hydrocarbon stream comprises the hydrocarbon stream passed to the oxygenate removal zone.

In a third aspect of the present invention, the invention may be characterized as a process for producing a sweetened hydrocarbon stream, in which the process comprises: removing acid gas from a liquid natural gas stream to form a scrubbed liquid natural gas stream; removing water from the scrubbed liquid natural gas stream in a dehydration zone to form a dehydrated liquid natural gas stream; separating the dehydrated liquid natural gas stream into at least one vapor stream and at least one liquid hydrocarbon stream; removing oxygenates from the at least one liquid hydrocarbon stream liquid hydrocarbon stream to form an oxygenate lean hydrocarbon stream; and, passing the oxygenate lean hydrocarbon stream to a sweetening zone to produce a sweetened steam.

In at least one embodiment, oxygenates are removed from the at least one liquid hydrocarbon stream liquid hydrocarbon stream by a water wash or an adsorbent material.

Additional objects, embodiments, and details of the invention are set forth in the following detailed description of the invention.

### DETAILED DESCRIPTION OF THE DRAWINGS

The drawing is a simplified process diagram in which the FIGURE shows a process for producing a sweetened hydrocarbon stream according to one or more embodiments of the present invention.

### DETAILED DESCRIPTION OF THE INVENTION

One or more process have been developed to minimize corrosion associated acidic species in the reactor vessel of a sweetening zone. In various embodiments, oxygenates, such as glycol, are removed from a liquid portion of a natural gas stream upstream of a sweetening zone. By including an oxygenate removal zone before the stream is passed to a reactor in the sweetening zone, there is a lower risk that oxygenates and the cobalt on the catalyst in the reactor will react to form acidic species which corrodes the vessel of the sweetening zone.

As shown in the FIGURE, an exemplary process according to the present invention for producing a sweetened stream from a liquid natural gas feed stream 10 is shown. Since hydrogen sulfide can interfere with downstream processing and extraction of mercaptans, the liquid natural gas feed stream 10 is passed to an acid gas removal zone 12 typically an amine scrubber to remove at least hydrogen sulfide.

For example, within the acid gas removal zone 12, carbonyl sulfide may be hydrolyzed to form hydrogen sulfide which is more easily removed from the system. Although not shown, the stream may then be sent to a separation zone, preferably an absorber unit, which may use an amine solvent (or other appropriate solvent), to remove carbon dioxide and hydrogen sulfide to produce a hydrogen sulfide rich stream 14 and a hydrogen sulfide lean hydrocarbon stream 16. The further processing of the hydrogen sulfide rich stream 14 is known, and it may, for example, be sent to a guard bed to remove any trace amounts of hydrogen sulfide, and then on to disposal or regeneration of the adsorbent.

From the acid gas removal zone 12, the hydrogen sulfide lean hydrocarbon stream 16 may be passed to a water wash 18. In the water wash 18, dissolved or entrained amine that has carried over from the acid gas removal zone 12 is removed.

From the water wash 18, a treated stream 20 is passed to a dehydration zone 22. The dehydration zone 22 typically includes a glycol dryer which can be used to remove water in the treated stream 20. The dehydration zone 22 may also include a low pressure separator (not shown), such a settling vessel, which allows various components to be separated based upon densities, phase, etc. These components are known in the art. More specifically, in a glycol dryer, a lean glycol stream is introduced into an absorber vessel. The treated stream is also introduced into the absorber vessel. The two streams may be co-current or countercurrent. For example, the lean glycol stream can be introduced at the top of the absorber vessel and flow downwards against the flow of the treated stream. The glycol will absorb water from the treated stream. A rich glycol stream can be recovered from the bottom of the absorber vessel. Since hydrocarbons may be contained in the rich glycol stream, the rich glycol stream can be passed to a settling vessel which is may be at a lower pressure than the absorber vessel. Therefore, any hydrocarbons in the rich glycol stream will flash and a hydrocarbon rich vapor stream can be recovered. The glycol rich stream can then be sent to a stripper column to remove water and regenerate the lean glycol stream which can be passed back to the absorber vessel and used to dehydrate the treated stream again. As discussed above it is believed that glycol from the glycol dryer is present in a dehydrated hydrocarbon stream 24 that is provided from the glycol dryer.

Returning to the FIGURE, the dehydrated hydrocarbon stream 24 may be passed from the dehydration zone 22 to a separation zone 26. As depicted, an example of the separation zone 26 comprises at least one separation column, and preferably a deethanizer column 28, a depropanizer column 30, and, a debutanizer column 32. As will be appreciated, these separation columns 28, 30, 32 operate to separate the dehydrated hydrocarbon stream 24 into one or more streams.

For example, in the deethanizer column 28, the dehydrated hydrocarbon stream 24 may be separated into a C₂− hydrocarbon stream 34 and a C₃₊ hydrocarbon stream 36. The C₃₊ hydrocarbon stream 36 may be passed to the depropanizer 30 and separated into a C₃ hydrocarbon stream 38 and a C₄₊ hydrocarbon stream 40. The C₄₊ hydrocarbon stream 40 may be passed to the debutanizer 32 and separated into a C₄ hydrocarbon stream 42 and a C₅₊ hydrocarbon stream 44. As mentioned above, the operation of these separations columns 28, 30, 32 are known in the art. It should be appreciated and understood that other configurations of separation columns or other separation zones may be used. In the prior art, a liquid hydrocarbon stream 46, such as the C₅₊ hydrocarbon stream 44, may pass to a sweetening zone 48 to convert mercaptans to disulfides.

However, as mentioned above, it has been discovered that trace amounts of acidic precursors are being passed with the liquid hydrocarbon stream 44, to the sweetening zone 48 and forming acid species that are corroding the vessels of the sweetening zone 48. While the trace amounts may be low, the amount of acid can increase overtime, thus accelerating any corrosion of the vessel. Accordingly, the present invention provides an oxygenate removal zone 50 upstream of the sweetening zone 48 to remove one or more acid precursors such as oxygenates from the liquid hydrocarbon stream 46 wherein the concentration of oxygenates in the liquid hydrocarbon stream 46 is at least about 1 ppm (by mass), preferably at least about 10 ppm (by mass).

As mentioned above, it is believed that one such oxygenate is glycol which may be present in the liquid hydrocarbon stream 46 based upon the glycol dryer of the dehydration zone 22. Accordingly, in one embodiment, the oxygenate removal zone 50 may comprise a water wash in which water is introduced to the liquid hydrocarbon stream 46. Based upon its affinity for glycol, water will pull the trace amounts of glycol from the stream. Typical operations for a water wash are contemplated to include a temperature range of about 26° C. to about 60° C. (80° F. to 140° F.). The water can be recirculated in the water wash at an amount of 10-25% by volume of the liquid hydrocarbon stream 46 passing into the water wash. More preferably, this is approximately a 20% recirculation rate.

In another embodiment, the oxygenate removal zone 50 may comprise an adsorbent zone. In the adsorbent zone, an adsorbent will adsorb oxygenates from the liquid hydrocarbon stream 46. For example, it is believed that an activated carbon may be used as an adsorbent for glycol.

It is most preferred that the oxygenate removal zone 50 comprises a regenerable adsorbent zone in which the adsorbent may be processed to separate the oxygenates from the liquid hydrocarbon stream 46. The spent adsorbent could then be regenerated in a regeneration zone, allowing the adsorbent to be recycled in the process. As will be appreciated, having a regenerable adsorbent zone will lower operating costs and minimize disposal concerns associated with adsorbent. One such contemplated regeneration process comprises a temperature swing desorption in which high temperature desorbent is used to strip off oxygenates from the adsorbent and provide a desorbent stream rich in oxygenates. While the desorbent stream rich in oxygenates may be disposed of, it is also contemplated, as shown in the FIGURE, that a desorbent stream rich in oxygenates 58 from the oxygenate removal zone 50 is combined with the product of the sweetening zone 48 (discussed below).

In any of these configurations, it is desired that the oxygenates in the liquid hydrocarbon stream 46 are maintained at a concentration level of less than 3% by weight, or preferably less than 1% by weight to produce an oxygenate lean hydrocarbon stream 52.

Returning to the FIGURE, the oxygenate lean hydrocarbon stream 52 is passed from the oxygenate removal zone 50 to the sweetening zone 48. The sweetening zone 48 includes a vessel 54 typically comprised of a carbon steel material. Inside of the vessel 54 is a catalyst which may comprise cobalt. When the vessel 54 of the sweetening zone 48 is operated under proper conditions, the catalyst converts the mercaptans to disulfides to produce a sweetened hydrocarbon stream 56. An exemplary process is described, for example, in U.S. Pat. No. 4,124,493.

More specifically, the sweetening zone 48 may utilize a process for converting mercaptans by contacting the liquid hydrocarbon stream 46 with a supported mercaptan oxidation catalyst at oxidation conditions in the presence of an alkaline reagent and a substituted ammonium halide. The catalyst employed can be any of the various catalysts known as effective to catalyze the oxidation of mercaptans contained in the liquid hydrocarbon stream 46 with the formation of polysulfide oxidation products. Said catalysts include the metal compounds of tetrapyridinoporphyrazine described in U.S. Pat. No. 3,980,582, e.g., cobalt tetrapyridinoporphyrazine; porphyrin and metaloporphyrin catalysts as described in U.S. Pat. No. 2,966,453, e.g., cobalt tetraphenylporphyrin sulfonate; corrinoid catalysts as described in U.S. Pat. No. 3,252,892, e.g., cobalt corrin sulfonate; chelate organometallic catalysts such as described in U.S. Pat. No. 2,918,426, e.g., the condensation product of an aminophenol and a metal of Group VIII; and the like. Metal phthalocyanines are a preferred class of mercaptan oxidation catalysts. The process is usually effected at ambient temperature conditions, although higher temperatures up to about 105° C. may be utilized. Pressures of up to about 6.9 MPa (1000 psi) or more are operable, although atmospheric or substantially atmospheric pressures are entirely suitable. Contact times equivalent to a liquid hourly space velocity of from about 0.5 to about 10 or more are effective to achieve a desired reduction in the mercaptan content of the liquid hydrocarbon stream 46, an optimum contact time being dependent on the size of the treating zone, the quantity of catalyst contained therein, and the character of the liquid hydrocarbon stream 46 being treated. As previously stated, sweetening of the liquid hydrocarbon stream 46 is effected by oxidizing the mercaptan content thereof to disulfides. Accordingly, the process is effected in the presence of an oxidizing agent, such as air, although oxygen or other oxygen-containing gas may be employed. The liquid hydrocarbon stream 46 may be passed upwardly or downwardly through a catalyst bed in the vessel 54 of the sweetening zone 48. The liquid hydrocarbon stream 46 may contain sufficient entrained air, or add air may be admixed with the liquid hydrocarbon stream 46 and charged to the sweetening zone 48 concurrently therewith. In some cases, it may be of advantage to charge the air separately to the sweetening zone 48 and countercurrent to the liquid hydrocarbon stream 46 separately charged thereto.

Although not required, some processes may separate the disulfides from the sweetened hydrocarbon stream 56. For example, a fractionation may be performed. As mentioned above, it is also contemplated that the sweetened hydrocarbon stream 56 is combined with the desorbent stream rich in oxygenates 58 from the oxygenate removal zone 50. Since the oxygenates bypassed the sweetening zone, the oxygenates cannot react with the catalyst in same, and thus, it is not likely that the acid species will be formed in the sweetened hydrocarbon stream 56.

As should be appreciated, by reducing the oxygenates that may be found in the portion of the liquid natural gas stream that is passed to the sweetening zone 48, the amount of acid species formed by the reaction of cobalt and oxygenates can be minimized. Thus, the corrosion of the vessel 54 in the sweetening zone 48 can be minimized. This will allow the vessel 54 of the sweetening zone 48 to be in operation longer and require less maintenance.

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understating the embodiments of the present invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A process for producing a sweetened hydrocarbon stream, the process comprising:
passing a liquid hydrocarbon stream to an oxygenate removal zone;
removing oxygenates from the liquid hydrocarbon stream in the oxygenate removal zone to form an oxygenate lean stream;
passing the oxygenate lean stream to a sweetening zone to convert mercaptans in the oxygenate lean stream in disulfide and produce a sweetened hydrocarbon stream.

2. The process of claim 1 wherein the oxygenates comprise at least one of glycols, poly glycols, organic acids, aldehydes, ketones, ethers, esters and alcohols, and the oxygenate removal zone comprises a water wash.

3. The process of claim 1 wherein the oxygenate removal zone comprises an adsorbent zone.

4. The process of claim 3 wherein the adsorbent zone comprises a regenerable adsorbent.

5. The process of claim 4 further comprising:
desorbing oxygenates from the regenerable adsorbent to provide a desorbent stream rich in oxygenates; and,
combining the desorbent stream rich in oxygenates with the sweetened hydrocarbon stream.

6. The process of claim 1 wherein the sweetening zone includes a catalyst.

7. The process of claim 6 wherein the catalyst includes cobalt.

8. The process of claim 1 wherein the liquid hydrocarbon stream comprises a C₅+ hydrocarbon stream.

9. The process of claim 1 wherein the liquid hydrocarbon stream comprises a bottoms stream from a separation zone.

10. The process of claim 1 wherein the liquid hydrocarbon stream is lean in disulfides.

11. A process for producing a sweetened hydrocarbon stream, the process comprising:
removing water from a liquid hydrocarbon stream in a glycol dehydration zone;
passing at least a portion of the liquid hydrocarbon stream from the dehydration zone to an oxygenate removal zone;
removing oxygenates from the liquid hydrocarbon stream in the oxygenate removal zone to form an oxygenate lean stream; and,
passing the oxygenate lean stream to a sweetening zone to reduce an amount of mercaptans in the oxygenate lean stream and produce a sweetened hydrocarbon stream, the sweetening zone comprising at least one vessel, each vessel containing a catalyst.

12. The process of claim 11 wherein each vessel of the sweetening zone comprises at least portion of a carbon steel material.

13. The process of claim 12 wherein the catalyst in each vessel of the sweetening zone comprises cobalt.

14. The process of claim 11, further comprising:
removing acid gas from the liquid hydrocarbon stream upstream of the sweetening zone.

15. The process of claim 14 wherein the oxygenate removal zone comprises a water wash.

16. The process of claim 14 wherein the oxygenate removal zone comprises an adsorbent zone.

17. The process of claim 16 wherein the adsorbent zone comprises a regenerable adsorbent.

18. The process of claim 17 further comprising:
regenerating the regenerable adsorbent.

19. The process of claim 13 further comprising:
separating the liquid hydrocarbon stream after the step of removing acid gas in a separation zone into at least a C₅+ hydrocarbon stream, wherein the C₅+ hydrocarbon stream comprises the hydrocarbon stream passed to the oxygenate removal zone.

20. A process for producing a sweetened hydrocarbon stream, the process comprising:
removing acid gas from a liquid natural gas stream to form a scrubbed liquid natural gas stream;
removing water from the scrubbed liquid natural gas stream in a dehydration zone to form a dehydrated liquid natural gas stream;
separating the dehydrated liquid natural gas stream into at least one vapor stream and at least one liquid hydrocarbon stream;
removing oxygenates from the at least one liquid hydrocarbon stream liquid hydrocarbon stream with water, an adsorbent material or both to form an oxygenate lean hydrocarbon stream; and,
passing the oxygenate lean hydrocarbon stream to a sweetening zone to produce a sweetened steam.
